(19) United States
(12) Patent Application Publication
(10) Pub. No.: US 2017/0121705 A1
(43) Pub. Date: May 4, 2017

(54) METHODS AND KITS FOR NUCLEIC ACID ISOLATION

(71) Applicant: BLOO SCIENTIFIC CORPORATION, Austin, TX (US)

(72) Inventors: MARIANNA GOLDRICK, Austin, TX (US); Masoud TOLOUE, Austin, TX (US)

(21) Appl. No.: 15/343,112

(22) Filed: Nov. 3, 2016

Related U.S. Application Data
(60) Provisional application No. 62/251,050, filed on Nov. 4, 2015.

(51) Int. Cl. C12N 15/10; C12Q 1/68

(52) U.S. Cl. CPC C12N 15/1013; C12Q 1/6806

(57) ABSTRACT

The current teachings disclose methods and kits for increasing the amount of nucleic acid that can be recovered from magnetic beads using elution solutions with high ionic strength.

### CROSS-REFERENCE TO RELATED APPLICATIONS

This application claims the benefit of U.S. Provisional Application Ser. No. 62/251050 filed Nov. 4, 2015, which is incorporated by reference in its entirety.

### FIELD

The current teachings generally relate to methods and kits for isolating nucleic acids from biological samples. More specifically, the disclosed methods and kits are suitable for increasing the quantity of nucleic acid recovered from magnetic beads using high ionic strength elution solutions.

### BACKGROUND

Many molecular biology analytical techniques require nucleic acid from one or more sample of interest. Typically, the nucleic acid is at least partially purified from the sample of interest prior to performing the analysis.

Methods for purifying nucleic acids typically include an initial step where a biological sample is disrupted in, or combined with, a reagent containing a chaotropic salt (to disrupt cellular membranes and denature nucleases), and optionally phenol or similar organic solvent. The resulting mixture is often referred to as a lysate. The lysate is often subjected to further processing steps that may include organic extraction and recovery of the nucleic acid by alcohol precipitation.

A widely used alternative to alcohol precipitation is solid phase extraction of nucleic acid onto various solid supports, for example but not limited to, silica and magnetic or paramagnetic particles. The magnetic particles, or beads, are typically either (a) coated with chemicals and/or polymers such as silica or nucleic acids, or (b) modified by linking the beads to chemically reactive moieties, including carboxyl and amino groups. Processing of biological samples for extraction of target molecules onto coated or otherwise modified magnetic beads typically includes disrupting the biological sample in a lysis solution comprising components to disrupt membranes and inactivate nucleases and/or proteases; adding magnetic beads to the disrupted sample; mixing the sample to disperse the beads in the liquid matrix; incubating the sample to allow adsorption/binding of the target molecule to the coated beads; attracting the beads by placing the vessel containing the sample in proximity to a magnet to allow the beads to migrate under the influence of the magnetic field to the side(s) of the vessel; removing the fluid from the vessel without disturbing the attracted beads; washing the beads by adding wash solution to the vessel with the attracted beads; optionally dispersing the beads in the wash solution and re-attracting them to the side of the vessel; removing the fluid; optionally repeating the wash step using wash solution(s) of same or different composition; drying the beads; and finally eluting the target molecule(s) from the beads by dispersing the beads in a low-ionic strength elution solution, incubating the sample to allow the target molecules to detach from the beads; re-attracting the beads to the side(s) of the vessel; and removing the eluate containing the target molecule(s).

Elution with low ionic strength buffers has been standard practice in molecular biology for many years. It is generally believed that low ionic strength solutions, including TE buffer and water, are needed to disrupt the molecular interactions, for example but not limited to salt bridges, which are believed to be responsible for causing nucleic acids to remain bound to solid supports used for solid-phase extraction. TE buffer typically comprises Tris-HCl or Tris base at 10 mM, pH 7-8, and EDTA at 0.1-1.0 mM. Published protocols related to elution of nucleic acids from solid-phase media (silica filters, silica beads, magnetic beads) teach that the elution is carried out by contacting the solid phase associated with the bound nucleic acid, with low-ionic strength solution, for example but not limited to, water, for a period of time sufficient for the bound nucleic acid to dissociate from the solid matrix and hence be recovered in the low ionic strength elution solution.

### SUMMARY

Contrary to the teachings that nucleic acids are efficiently eluted in low ionic strength solutions, the inventors were surprised to discover that recovery of nucleic acid from certain magnetic beads is more efficient when the elution step is carried out using an elution solution having a relatively high ionic strength. Efforts to maximize yields of nucleic acids extracted using magnetic beads led us to test elution solutions having various concentrations of sodium chloride, which in turn led to the surprising discovery that elution in higher ionic strength elution solution can increase the amount of nucleic acid recovered substantially. The inventors were also surprised to discover that this affect does not appear to extend to the related salts, potassium chloride and sodium acetate.

According to certain disclosed methods, a biological sample is combined in a reaction vessel with a nucleic acid extraction reagent and magnetic beads. The vessel is incubated under conditions suitable for at least some of the nucleic acid present in the reaction mixture to bind or adhere to the magnetic particles. The vessel is then placed in the proximity of a magnetic field for a time sufficient for the magnetic beads to migrate under the influence of the field to at least one surface of the vessel, for example but not limited to, the side(s) adjacent to, or in the proximity to, the magnetic field. The nucleic acid extraction reagent is substantially removed, leaving at least some of the magnetic beads comprising nucleic acid in the vessel. The magnetic field is removed from the vessel so that the particles in the vessel are no longer under the influence of the field. The particles are then re-suspended in an elution solution comprising at least 20 mM sodium chloride (NaCl) and the vessel is incubated under conditions suitable for at least some of the nucleic acid to be eluted or released from the magnet particles. The vessel is again placed in the proximity of a magnetic field for a time sufficient for the magnetic beads to migrate under the influence of the field to at least one surface of the vessel. The elution solution comprising at least some of the nucleic acid from the sample is substantially removed, thereby isolating the nucleic acid from the sample in the elution solution.

According to certain embodiments, the elution solution comprising the isolated nucleic acid is not removed from the beads, but is stored with the beads.

According to certain embodiments, the beads are separated from the nucleic acid extraction reagent, the wash solution(s) and the elution solution by filtration rather than using a magnetic field.

In certain method embodiments, after the nucleic acid extraction reagent is removed, the magnetic beads are washed using a wash solution. In certain embodiments, a wash solution is combined with the magnetic beads in a vessel. The vessel with the magnetic beads and wash solution may optionally be removed from the proximity of the magnetic field and the magnetic beads may be suspended in the wash solution. If the beads are resuspended, the vessel is then placed in the proximity of a magnetic field for a time sufficient for the magnetic beads to migrate under the influence of the field to at least one surface of the vessel. The wash solution is then substantially removed, leaving at least some of the magnetic beads comprising nucleic acid in the vessel. This wash step may be repeated multiple times before suspending the magnetic beads in the elution solution.

In certain embodiments, the elution solution comprises between at least 20 mM NaCl and at least 2 M NaCl, for example but not limited to at least 20 mM NaCl, at least 50 mM NaCl, at least 75 mM NaCl, at least 100 mM NaCl, at least 125 mM NaCl, at least 250 mM NaCl, at least 500 mM NaCl, at least 1 M NaCl, and at least 2 M NaCl. The skilled artisan will appreciate that any concentration of NaCl between 20 mM and 2 M is within the scope of the current teachings. Thus, it is to be understood that all concentrations of NaCl between 20 mM and 2 M, although not individually recited, are within the scope of the instant disclosure and are to be considered recited herein throughout the entire 20 mM to 2 M range.

In certain exemplary embodiments, nucleic acid is purified from a biological sample, for example but not limited to, urine or plasma, by solid-phase extraction onto magnetic beads, and eluted from the beads in a high ionic strength elution solution comprising at least 20 mM sodium chloride.

According to certain embodiments, methods provided for obtaining nucleic acid from a biological sample, wherein the biological sample is combined with a solid support, for example but not limited to magnetic beads, and optionally a nucleic acid extraction reagent and the reaction mixture is incubated under conditions suitable for at least some of the nucleic acid from the sample to bind to the magnetic beads. The beads are separated from the reaction mixture, for example but not limited to using a magnetic field or other separation technique such as filtration, including vacuum filtration, or centrifugation. The reaction mixture is removed and the beads are re-suspended in a high ionic strength elution solution and incubated under conditions suitable for the bound nucleic acid to be released from the beads into the elution solution. The beads are separated from the elution solution, for example but not limited to using a magnetic field or other separation technique such as filtration, including vacuum filtration, or centrifugation. According to certain embodiments, the beads comprising nucleic acid from the sample are washed at least once using wash solution. In these embodiments, after the beads are separated from the wash solution, for example but not limited to, by using a magnetic field or other separation technique such as filtration, including vacuum filtration, or centrifugation. In certain embodiments, the washed beads are re-suspended in a high ionic strength elution solution and incubated under conditions suitable for at least some of the bound nucleic acid to be released from the beads into the elution solution. In certain embodiments, the high ionic strength elution solution is separated from the solid support, for example but not limited to using a magnetic field or other separation technique such as filtration, including vacuum filtration, or centrifugation.

In certain embodiments, the elution solution comprising nucleic acid obtained from a biological sample is not initially removed from the vessel, but may be stored for later use or used in a subsequent analytical technique. Thus, the elution solution comprising the nucleic acid obtained from the biological sample is left in the vessel with the magnetic beads.

In certain embodiments, at least some of the elution solution comprising nucleic acid obtained from the sample is removed from the vessel, thereby isolating nucleic acid from the sample. For example but not limited to, one may remove aliquots of elution solution from the vessel for subsequent analysis, while leaving at least some, and in certain circumstances the majority, of the elution solution in the vessel.

### BRIEF DESCRIPTION OF THE FIGURES

These and other features and advantages of the current teachings will become better understood with regard to the following description, appended claims, and accompanying figures. The skilled artisan will understand that the figures, described below, are for illustration purposes only. The figures are not intended to limit the scope of the disclosed teachings in any way.

FIG. 1: depicts an agarose gel loaded with PCR amplicons (human 28S ribosomal RNA) obtained using aliquots of nucleic acid obtained by eluting with either high ionic strength elution solutions or low ionic strength elution solutions, as described in Example 1. The gel was stained with ethidium bromide.

FIGS. 2A-2B: FIG. 2A shows an electropherogram obtained using nucleic acid recovered using an elution solution comprising no NaCl, as described in Example 3. FIG. 2B shows an electropherogram obtained using nucleic acid recovered using an elution solution comprising 75 mM NaCl, as described in Example 3.

### DETAILED DESCRIPTION OF CERTAIN EXEMPLARY EMBODIMENTS

It is to be understood that both the foregoing general description and the following detailed descriptions are illustrative and exemplary only and are not intended to limit the scope of the disclosed teachings. The section headings used herein are for organizational purposes only and are not to be construed as limiting the subject matter of the disclosed teachings.

In the Summary above, the Detailed Description, the accompanying Figures, and the claims below, reference is made to particular features (including method steps) of the current teachings. It is to be understood that the disclosure in this specification includes possible combinations of such particular features. For example but not limited to, where a particular feature is disclosed in the context of a particular embodiment of the current teachings, or a particular claim, that feature can also be used, to the extent possible, in combination with and/or in the context of other particular embodiments, and in the current teachings in general.

Where reference is made to a method comprising two or more combined steps, the defined steps can be performed in any order or simultaneously (except where the context excludes that possibility), and the method may include one or more additional steps which are carried out before any of the defined steps, between two of the defined steps, or after all of the defined steps (except where the context excludes that possibility).

In this specification, certain U.S. patents, U.S. patent applications, and other documents may have been incorporated by reference. The text of such U.S. patents, U.S. patent applications, and other materials is, however, only incorporated by reference to the extent that no conflict exists between such text and the description and drawings set forth in this specification. In the event of such conflict, then any conflicting material in any incorporated by reference U.S. patents, U.S. patent applications, and other materials is specifically not incorporated by reference in this specification.

Definitions

The term “biological fluid” means fluids that may be produced by living organisms. Examples of biological fluids include, without limitation, serum and plasma derived from blood; urine; saliva; cerebrospinal fluid; synovial fluid; pleural effusions; breast milk; and culture media used to grow and propagate eukaryotic and prokaryotic cells.

Analyses carried out on biological fluids from which the cells have been removed, for example but not limited to by centrifugation, are generally more informative for certain types of diagnostic tests, compared to analyzing the unfractionated fluid which may additionally contain substantial quantities of nucleic acid released from cells in the fluids. This is because the nucleic acid of interest may be present in minute quantities, and in unfractionated biological fluid it would be diluted by the vastly higher amounts of nucleic acid present in the cells. Unfractionated whole blood is comprised of plasma, white blood cells, and red blood cells. Most of the nucleic acid present in whole blood is in the white blood cells. The plasma comprises soluble proteins and other biological molecules including fragments of DNA that originate from cells during normal physiological processes such as apoptosis and autophagy. Malignant cells can also release DNA fragments into the plasma, by processes including direct lysis and necrosis. The DNA released by malignant cells into the circulation is sometimes referred to as circulating tumor DNA or ctDNA. Circulating tumor DNA may have diagnostic value; analysis of the informative DNA released from malignant cells into the circulation is facilitated by removing the DNA from the non-malignant white blood cells, which would otherwise dilute the signal from the DNA derived from malignant cells. The concept of monitoring the progression of malignant disease by analysis of cell-free DNA or ctDNA recovered from plasma is known as “liquid biopsy”. Liquid biopsy has advantages over traditional biopsy of solid tumors, since the sample (plasma) can be recovered without surgical intervention. Another example of clinical interest in the use of cell-free DNA is for non-invasive prenatal diagnostics (NIPD). DNA of fetal origin is released into the maternal circulation and can be used for detection of fetal genetic abnormalities such as trisomy of Chromosome 21, the cause of Down syndrome. Removal of the white blood cells from the maternal blood sample prior to genetic analysis allows more sensitive detection of fetal abnormalities.

The term “biological sample” is used in a broad sense and includes cells (including in vitro cell cultures), tissue, organs, and biological fluids, for example but not limited to blood, lymph, cerebrospinal fluid, synovial fluid, pleural fluid, obtained from an organism (either eukaryotic or prokaryotic), directly or indirectly.

The term “cell-free” when used in the context of biological fluid refers to either (a) a biological fluid that does not initially contain cells or (b) biological fluids from which cells that were initially in such fluids have been removed, for example but not limited to, by centrifugation, filtration, or fractionation. Examples of cell-free biological fluids include, amniotic fluid, plasma, or urine which may be first centrifuged to pellet any cells in such fluids prior to use as a source of cell-free nucleic acid. The skilled artisan will appreciate that by first removing cells from certain biological fluids, one avoids contaminating the nucleic acid that is present in the biological fluid in a soluble, free form (cell-free nucleic acid) with the nucleic acid contained in whole cells or cell components that may also be present in the same fluid prior to separation. The nucleic acid obtained from such cell-free biological fluids is sometimes referred to as cell-free nucleic acid, cell-free DNA (cfDNA), and similar terms.

As used herein, the term “comprising”, which is synonymous with “including”, and cognates of each (such as comprises and includes), is inclusive or open-ended and does not exclude additional unrecited components, elements, or method steps, that is other components, steps, etc., are optionally present. For example but not limited to, an article “comprising” components A, B, and C may consist of (that is, contain only) components A, B, and C; or the article may contain not only components A, B, and C, but also one or more additional components.

The term “elution solution” means a liquid or gel that is used to remove (elute) nucleic acid from a solid support, for example but not limited to beads (such as magnetic, paramagnetic, polystyrene, complex carbohydrates such as Sepharose, Sephadex, DEAE dextran), and the like, including modified or derivatized versions thereof. Solid supports include beads in a column or centrifuge tube, and surfaces (such as solid or flexible supports such as nitrocellulose, silica filters, and microarrays). Exemplary elution solutions include aqueous solutions that may comprise a buffer such as Tris-Cl, Tris-base, HEPES, sodium phosphate, or the like (often present at concentrations of 20 mM or less) and that may further comprise a chelating agent such as EDTA (ethylenediaminetetraacetic acid), the sodium salt of EDTA, EGTA, generally present at concentration less than 5 mM. Certain elution solutions known in the art comprise less than 15 mM salt.

The term “high ionic strength”, when used in reference to elution solutions, means a solution comprising at least 20 mM salt, such as NaCl. The high ionic strength elution solutions of the current teachings typically comprise salt concentrations in the range of 20 mM to 2 M, including all concentrations within that range.

The term “nucleic acid extraction reagent” refers to a reagent comprising components to disrupt cell membranes and/or disrupt proteins and/or inactivate nucleases and/or promote binding of nucleic acid to solid supports such as magnetic beads. Said nucleic acid reagents typically comprise chaotropic salts such as guanidinium thiocyante, guanidinium chloride, or urea; and may contain detergents or other surfactants, volume-excluding agents such as polyethylene glycol, and proteases such as Proteinase K. Nucleic acid extraction reagents are typically liquids, but may be prepared as solids and subsequently dissolved in water or other buffers prior to use.

The term “vessel” is used in a broad sense herein and is intended to encompass any container, regardless of size or shape, which is suitable for use in the disclosed methods and kits. Exemplary vessels include centrifuge tubes (such as 1 mL, 2 ml, 15 mL, and 50 mL tubes), beakers and flasks, and test tubes. For clarity, as used herein, terms such as “side(s) of the vessel”, “at least one surface of the vessel”, and similar terms are intended in a broad context and include, curvilinear sides or bottom of a centrifuge tube, microfuge tube, or round bottom tube.

The term “wash solution” means a fluid added to a nucleic acid bound to a solid support such as magnetic beads, especially after attraction of the magnetic beads to a magnetic field, which is used to remove impurities from the bound nucleic acids and/or solid support. Examples of common wash solutions are fluids comprising alcohols such as ethanol, salts such as sodium chloride, typically present at concentration of 100 mM-1 M, and water, typically nuclease free. Exemplary wash solutions comprise 75% ethanol and 500 mM NaCl in nuclease-free water; and 75% ethanol and 250 mM NaCl in nuclease-free water. It is to be appreciated that wash solutions typically have high ionic strength, which presumably minimizes elution of nucleic acid bound to the solid phase substrate material being washed. In light of the fact that such wash solutions typically have high ionic strengths, our discovery that an elution solution of high ionic strength leads to better recovery of nucleic acids was truly an unexpected result, as the inventors had previously assumed that high ionic strength should correlate to increased retention of bound nucleic acid rather than increased elution.

Magnetic beads for nucleic acid isolation are commercially available from many different vendors. Information about the properties of commercially available magnetic beads used for nucleic acid isolation, including their composition, physical properties, and especially their chemical coatings and surface modifications, is typically proprietary. Desirable properties for magnetic beads used for nucleic acid purification are that they are easily dispersed in liquid matrices (including nucleic acid extraction reagent, wash solutions, and elution solution); they are rapidly and completely attracted to magnetic fields, and easily dispersed from the side(s) of the vessels in the absence of a magnetic field; they have a high binding capacity for nucleic acids; and that bound nucleic acids can be efficiently eluted from the beads during the elution step.

### Certain Exemplary Kits

In certain embodiments, kits are provided to expedite the performance of various disclosed methods. Kits serve to expedite the performance of certain method embodiments by assembling two or more reagents and/or components used in carrying out certain methods. Kits may contain reagents in pre-measured unit amounts to minimize the need for measurements by end-users. Kits may also include instructions for performing one or more of the disclosed methods. In certain embodiments, at least some of the kit components are optimized to perform in conjunction with each other. Typically, kit reagents may be provided in solid, liquid, or gel form.

In certain embodiments, kits for isolating nucleic acid from a solid support are provided. In certain embodiments, kits comprise a nucleic acid extraction reagent; a wash solution; and a high ionic strength elution solution. In certain embodiments, kits further comprise a solid support, including magnetic beads. In certain embodiments, kits further comprise at least one vessel.

### Certain Exemplary Methods

### EXAMPLE 1

### Higher Yields of Nucleic Acid were Recovered from Preps Eluted in Elution Solution Containing Sodium Chloride, as Determined using Qubit Assay

Nucleic acid was extracted from 0.9 ml of blood plasma from each of 2 donors using the NextPrep-Mag™ cfDNA Isolation kit (Bioo Scientific, cat #3825) according to the manufacturer's protocol except that for some samples, the elution solution in the kit was supplemented with sodium chloride to the final concentrations indicated in TABLE 1. For samples eluted with no sodium chloride, two extractions were carried out, one using non-stick (also called “low-binding”) microfuge tubes (DNA LoBind tube, Eppendorf Cat no 022431021) to assess whether this type of tube would result in higher recovery of nucleic acid. Nucleic acid was eluted from the magnetic beads using 100 μL elution solution for all samples. The beads were incubated in the elution solution for five minutes at room temperature prior to being subjected to a magnetic field to separate the beads from the fluid comprising the isolated nucleic acid. The nucleic acid concentrations in each of the recovered eluates were determined using the Qubit® High Sensitivity DNA assay (Agilent Technologies cat #5067-4626) at 1:20 dilutions of each recovered eluate. TABLE 1 shows (a) the concentration of nucleic acid obtained from each recovered eluate and (b) the fold-increases in recovery of nucleic acid using elution solution supplemented with sodium chloride, compared to the baseline samples eluted with no sodium chloride. These results demonstrate that as the ionic strength of the elution solution increases (based on NaCl concentration), the amount of nucleic acid recovered from the magnetic beads also increases. Thus, contrary to current practices, nucleic acid yields are substantially increased using high ionic strength elution solutions.

TABLE 1

| | | Concentration of | Fold increase |
|---|---|---|---|
| | [NaCl] in | recovered nucleic | compared to baseline |
| Plasma | Elution | acid at 1:20 dilution | condition (0 NaCl |
| Donor | Solution | (ng/mL) | in Elution Solution) |
| 1 | 0 | 10.0 | — |
| 1 | 25 mM | 17.2 | 1.7 X |
| 1 | 50 mM | 27.6 | 2.8 X |
| 1 | 100 mM | 36.4 | 3.6 X |
| 2 | 0 | 4.2 | — |
| 2 | 25 mM | 5.4 | 1.3 X |
| 2 | 50 mM | 11.0 | 2.6 X |
| 2 | 100 mM | 13.0 | 3.1 X |

### EXAMPLE 2

### PCR Assay Showing Higher Yields of Nucleic Acid in Preps Eluted in Elution Solution Containing Sodium Chloride

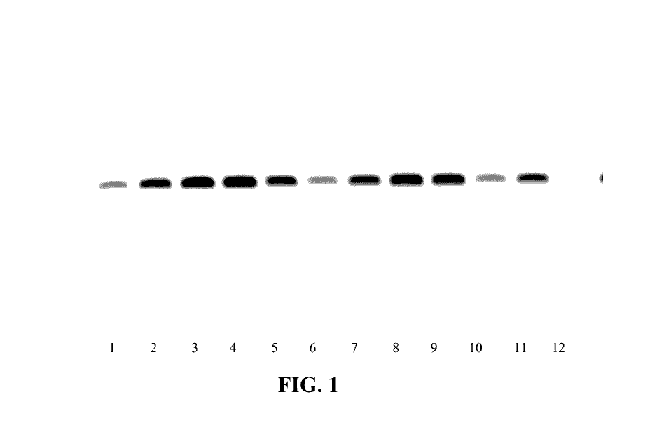

The recovered eluates described in Example 1 were used as input for PCR reactions mixtures that contained primers designed to amplify a 170 by region of DNA encoding human 28S ribosomal RNA. Four microliters of nucleic acid recovered from a given preparation were used as input for separate 20 μL PCR reactions. Since the nucleic acid was recovered in the same volume of elution solution for each preparation, each PCR comprised an equal proportion of the recovered nucleic acid used as input. The PCR reaction was run for 28 cycles and the products analyzed by agarose gel electrophoresis and the amplicons were detected by ethidium bromide staining, as shown in FIG. 1. The sodium chloride concentration in the solution used to elute the nucleic acid in each preparation used as input for the lanes corresponding to each PCR is shown in TABLE 2 below. This experiment demonstrated higher yields of products amplified from preparations of nucleic acid eluted in solutions containing sodium chloride. The experiment also included samples processed in special “low-binding” microfuge tubes, which are treated to minimize binding of nucleic acids.

TABLE 2

| Lane in gel in | | |
|---|---|---|
| FIG. 1 | Donor | [NaCl] used for elution step |
| 1 | 1 | 0 mM |
| 2 | 1 | 25 mM |
| 3 | 1 | 50 mM |
| 4 | 1 | 100 mM |
| 5 | 1 | 0 mM (in low-bind microfuge tube) |
| 6 | 2 | 0 |
| 7 | 2 | 25 mM |
| 8 | 2 | 50 mM |
| 9 | 2 | 100 mM |
| 10 | 2 | 0 (in low-bind microfuge tube) |
| 11 | NA | Positive control |
| 12 | NA | Negative control |

### EXAMPLE 3

### Higher Yields of Nucleic Acid Recovered Using Elution Solution Containing Sodium Chloride, as Determined by Microfluidic Analysis

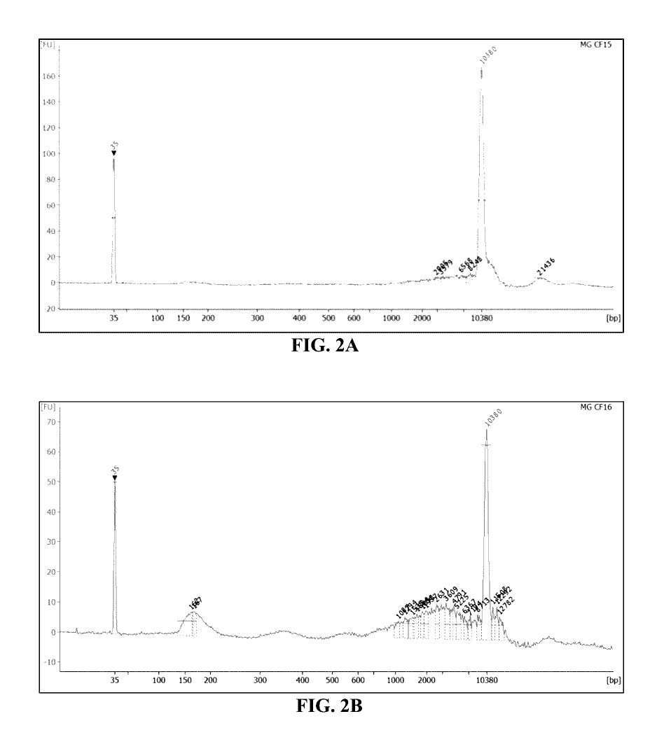

Nucleic acid was extracted from duplicate 0.9 ml samples of human plasma, each of which was spiked with 2 μL of nucleosomes from HEK293 cells (AMSBIO, cat #52015, Abingdon OX14 4SE, UK). The nucleosomes were added to increase the amount of nucleic acid to levels more easily detectable using a microfluidic assay. Nucleosomes were used for this purpose instead of purified nucleic acid, because the nucleosomes more closely reflect the endogenous nucleic acid found in human plasma, which is believed to derive from nucleosomes and other protein-associated and membrane-associated particles. Nucleic acid was extracted using the NextPrep-Mag™ cfDNA Isolation kit (Bioo Scientific, cat #3825) except that for one of the two preps, the elution solution in the kit was supplemented with sodium chloride to a final concentration of 75 mM. Nucleic acid was eluted in a volume of 50 μL elution solution and the recovered eluates were analyzed on an Agilent 2100 Bioanalyzer High Sensitivity DNA Chip (Agilent Technologies, Santa Clara Calif.). Nucleic acid is quantitatively detected in this assay by binding to a fluorescent dye. The y-axis in the graphs is labeled in FU, Fluorescent Units. The height of the peaks between the 35 bp and the 10380 bp lower and upper molecular weight markers in the graphs reflects the yields of nucleic acid. The x-axis is labeled by (basepairs) and the position of the peaks along the x-axis reflects the size of the nucleic acid molecules being detected. FIG. 2A shows the electropherogram of the sample eluted in elution solution containing no sodium chloride and FIG. 2B shows the electropherogram of the sample eluted in elution solution containing 75 mM sodium chloride. The sample eluted without sodium chloride is essentially undetectable, while the sample eluted in 75 mM sodium shows peaks of nucleic acid at about 150 bp, as expected for nucleic acid derived from nucleosomes, and also a peak of higher-molecular weight nucleic acid that may reflect the presence of cellular nucleic acid present in the plasma.

### EXAMPLE 4

### Higher Recovery of Nucleic Acid Using Elution Solution Comprising Sodium Chloride Compared to an Elution Solution not Containing Sodium Chloride

Nucleic acid was extracted from 0.9 ml of blood plasma from a single donor using the NextPrep-Mag™ kit except that (a) different magnetic beads were used and (b) for some samples, the elution solution in the kit was supplemented with sodium chloride to a final concentration of 75 mM. Nucleic acid was eluted from the various beads using 75 μL elution solution, and concentrations were determined using Qubit® High Sensitivity DNA assay (Agilent Technologies cat #5067-4626) at 1:20 dilutions of each recovered eluate, as described in the manufacturer's protocol.

The source of the magnetic beads, the concentration of sodium chloride used to elute the nucleic acid from the magnetic beads, and the concentrations of nucleic acid obtained for each prep are shown in TABLE 3. Increased recovery of nucleic acid using an elution solution supplemented with NaCl to a final concentration of 75 mM NaCl was seen for Preps 2, 4, and 6. Additionally, not all the magnetic beads tested showed improved recovery of nucleic acid when eluted in solution containing sodium chloride. Two examples of such magnetic beads (those used in Preps 8 and 10) are also included in TABLE 3.

TABLE 3

| | | [NaCl] in elution solution. | Concentration |
|---|---|---|---|
| | | Baseline is the elution | of nucleic |
| | Source of | solution included in the | acid recovered |
| | Magnetic | NextPrep-Mag ™ | (at 1:20 |
| Prep # | beads | cfDNA Isolation kit | dilution) |
| 1 | Bioo Scientific * | Baseline (no NaCl) | 3.07 ng/mL |
| 2 | Bioo Scientific * | Baseline + 75 mM NaCl | 23.4 ng/mL |
| 3 | SeraMag Inc. | Baseline (no NaCl) | 8.04 ng/mL |
| | carboxylated | | |
| | #651521050 | | |
| 4 | SeraMag Inc. | Baseline + 75 mM NaCl | 22.3 ng/mL |
| | carboxylated | | |
| | #651521050 | | |
| 5 | MagSphere Inc. | Baseline (no NaCl) | Too low to |
| | carboxylated | | read |
| | #MCA800NM | | |
| 6 | MagSphere Inc. | Baseline + 75 mM NaCl | 1.78 ng/mL |
| | carboxylated | | |
| | #MCA800NM | | |
| 7 | Sphero-Mag Inc. | Baseline (no NaCl) | 29.4 ng/mL |
| | #CMX-10-10 | | |
| 8 | Sphero-Mag Inc. | Baseline + 75 mM NaCl | 19.8 ng/mL |
| | #CMX-10-10 | | |
| 9 | Promega Corp. | Baseline (no NaCl) | 7.11 ng/mL |
| | MagneSil | | |
| 10 | Promega Corp. | Baseline + 75 mM NaCl | 7.38 ng/mL |
| | MagneSil | | |

* these beards were produced essentially as described in: Stöber, W.; Fink, A.; Bohn, E. J. Colloid. Interface Sci. 1968, 26, 62, which is incorporated by reference in its entirety.

### EXAMPLE 5

### Comparison of Elution Results using Related Salts

Cell-free DNA (cfDNA) was extracted from 0.9 ml of plasma using the Bioo NextPrep-Mag™ kit except that for various preps, the elution solution was supplemented with either NaCl, potassium chloride (KCl; i.e., same anion, different cation), or with sodium acetate (CH₃COONa; NaOAc; i.e., same cation, different anion). In each case the final concentration of salt added to the elution solution was 75 mM. The cfDNA was eluted in a volume of 65 μL with incubation at room temperature for five minutes. Yields were determined by Qubit assay on 1:20 dilutions of the recovered cfDNA, as described above. Surprisingly, the amount of nucleic acid eluted from the beads when the elution solution comprised either 75 mM KCl or 75 mM NaOAc did not differ significantly from the amount eluted when no additional salt was added, as seen in TABLE 4. In contrast, the amount of nucleic acid eluted from beads when the elution solution comprised 75 mM NaCl was significantly greater compared to the amount eluted with the NextPrep-Mag· kit's elution solution (no added salt).

TABLE 4

| | | Concentration of eluted |
|---|---|---|
| | | cfDNA (Qubit ® value at |
| Prep | Salt added to elution solution | 1:20 dilution) |
| A | None | 16.4 |
| B | NaCl | 25.1 |
| C | KCl | 15.2 |
| D | NaOAc | 16.5 |

While the above examples were performed using cell-free blood plasma, it is to be understood that similar results would be obtained when using other biological samples. Thus, it is contemplated that the advantages of high ionic strength elution solution, particularly those comprising between 20 mM and 2 M NaCl, will also be observed when purifying nucleic acid, such as cfDNA from other biological fluids, such as urine, cerebrospinal fluid, serum, synovial fluid, etc. The same result is also expected to be the case for cfDNA extracted from culture media used to grow eukaryotic and prokaryotic cells. Thus, the person of skill in the art will appreciate that such applications are within the scope of the current teachings.

The skilled artisan will also appreciate that, although the examples demonstrate enhanced recovery of DNA from solid supports through the use of high ionic strength elution solutions, similar results may be obtained when isolating RNA from biological solutions, including cell-free solutions and culture media from prokaryotic and eukaryotic cell cultures.

For example but not limited to, plasma or other biological sample may be combined with a nucleic acid extraction reagent comprising protease(s) such as Proteinase K to degrade RNases present in biological samples, and magnetic beads. Following certain disclosed methods, after the binding and washing steps, the RNA bound to the magnetic beads may be efficiently eluted using a high ionic strength elution solution, as disclosed herein. The protease digestion step may be carried out in the presence of detergents such as SDS, and/or carried out at elevated temperature, to increase the efficiency of degradation of RNase(s). These conditions for protease digestion are well-known to those skilled in the art.

RNA may be recovered from biological fluids using phenol-based reagents such as TRIzol® or TRIzol®-LS (Thermo Fisher Scientific, cat #10296010, #10296028, respectively). The aqueous phase recovered during use of these products can be mixed with magnetic beads and optionally with other reagents, and incubated under conditions effective for the binding of RNA to the magnetic beads. After subsequent steps including washing, attraction to magnet, removal of fluids, drying of beads, etc., the RNA may be eluted from the beads using a high ionic strength elution solution, as disclosed herein.

Although the disclosed teachings have been described with reference to various applications, methods, and compositions, it will be appreciated that various changes and modifications may be made without departing from the teachings herein. The foregoing examples are provided to better illustrate the present teachings and are not intended to limit the scope of the teachings herein. Furthermore, various presently unforeseen or unanticipated alternatives, modifications, variations or improvements therein may be subsequently made by those skilled in the art which are also intended to be encompassed by the following claims. Certain aspects of the present teachings may be further understood in light of the following claims.

What is claimed is:

1. A method for isolating nucleic acid from a biological sample comprising:
combining a biological sample, a nucleic acid extraction reagent, and magnetic particles in a vessel;
incubating the vessel under conditions suitable for binding the nucleic acid to the magnetic particles;
placing the vessel in proximity to a magnetic field to allow the magnetic particles to migrate in the magnetic field to at least one surface of the vessel;
substantially removing the nucleic acid extraction reagent from the vessel;
removing the magnetic field from the proximity of the vessel;
resuspending the magnetic beads in an elution solution comprising at least 20 mM sodium chloride (NaCl) to form an elution composition;
incubating the elution composition under conditions suitable for releasing at least some of the nucleic acid from the magnetic particles into the solution;
placing the vessel in proximity to a magnetic field to allow the magnetic particles to migrate in the magnetic field to at least one surface of the vessel; and
removing at least some of the elution solution comprising at least some of the released nucleic acid from the vessel, thereby isolating nucleic acid from the sample.

2. The method of claim 1, wherein the elution solution comprises at least 50 mM NaCl.

3. The method of claim 1, wherein the elution solution comprises at least 100 mM NaCl.

4. The method of claim 1, wherein the biological sample is a cell-free biological fluid.

5. The method of claim 4, wherein the cell-free fluid is plasma, serum, or urine.

6. The method of claim 1, wherein the nucleic acid is DNA.

7. The method of claim 1, further comprising:
resuspending the magnetic particles in the vessel in a wash solution prior to adding the elution solution;
placing the vessel in proximity to a magnetic field to allow the magnetic particles to migrate in the magnetic field to at least one surface of the vessel; and
substantially removing the wash solution from the vessel.

8. The method of claim 7, wherein the elution solution comprises at least 50 mM NaCl.

9. The method of claim 8, wherein the elution solution comprises at least 100 mM NaCl.

10. A kit for isolating nucleic acid comprising a nucleic acid extraction reagent; at least one wash solution; and a high ionic strength elution solution.

11. The kit of claim 10, wherein the high ionic strength elution solution comprises between 20 mM and 125 mM NaCl.

12. The kit of claim 11, wherein the high ionic strength elution solution comprises about 50 mM NaCl.

13. The kit of claim 10, further comprising at least one vessel.

14. The kit of claim 10, further comprising a solid support.

15. The kit of claim 13, wherein the solid support comprises magnetic beads.

16. The kit of claim 14, wherein the high ionic strength elution solution comprises between 20 mM and 125 mM NaCl.
